(19) United States
(12) Patent Application Publication
(10) Pub. No.: US 2012/0039880 A1
(43) Pub. Date: Feb. 16, 2012

(54) FRAGMENTATION RESISTANT IgG1 Fc-CONJUGATES

(75) Inventors: Boxu Yan, San Diego, CA (US); Zhonghua Hu, Kenmore, WA (US); Gerd Richard Kleeman, Mercer Island, WA (US); Zachary Adam Yates, Bothell, WA (US); Hongxing Zhou, Bellevue, WA (US)

(73) Assignee: AMGEN INC., Thousand Oaks, CA (US)

(21) Appl. No.: 13/265,833

(22) PCT Filed: Apr. 21, 2010
(86) PCT No.: PCT/US10/31933

§ 371 Date: Oct. 21, 2011

Related U.S. Application Data
(60) Provisional application No. 61/171,393, filed on Apr. 21, 2009.

(51) Int. Cl. A61K 39/395; C07H 21/00; A61P 43/00; C12N 5/10; C12P 21/00; C07K 16/00; C12N 15/63

(52) U.S. Cl. 424/133.1; 530/387.3; 536/23.53; 536/23.4; 435/320.1; 435/328; 435/69.6

(57) ABSTRACT

The present invention provides compositions and methods relating to human IgG1 and IgG3 Fc-conjugates which are resistant to free-radical mediated fragmentation and aggregation. The present invention also provides compositions and methods for making the Fc-conjugates of the invention.

### CROSS REFERENCE TO RELATED APPLICATIONS

This application claims the benefit under 35 U.S.C. 119(e) of U.S. patent application number 61/171,393 filed Apr. 21, 2009 which is incorporated herein by reference.

### FIELD OF THE INVENTION

The present invention relates to immunoglobulins for use in therapeutic and diagnostic applications which are resistant to fragmentation from reactive oxygen species.

### BACKGROUND OF THE INVENTION

Human immunoglobulin (IgG) molecules consist of two identical copies of light chains (LCs) and heavy chains (HCs). An inter-chain disulfide bond between the LC and HC connects them to form a half antibody; the HCs of the two identical copies of the half antibody are connected by disulfide bonds in a so-called hinge sequence to form the native antibody. The human IgG1 hinge sequence includes two pairs of cysteine (Cys) residues that can form two separate disulfide bonds. However, it has been suggested that only a single hinge disulfide is necessary for complement-mediated lysis and antibody-dependent cell-mediated cytotoxicity and phagocytosis. Michaelsen, T. E. et al., Proc. Natl. Acad. Sci. USA 91: 9243-9247, 1994. Only a single inter-heavy chain disulfide bond has been observed in the crystal structure of IgG1 b12—the authors suggested that the broken disulfide bond may be dynamic or the result of synchrotron radiation damage. Stanfield, R. et al., Science 248: 712-719, 1990; Saphire, E. et al., J. Mol. Biol. 319: 9-18, 2002; Weik, M. et al., Proc. Natl. Acad. Sci. USA 97: 623-628, 2000. In fact, both oxidized and reduced conformations for a solvent-exposed single cysteine pair in a crystal structure have been noted. Burling, F. T. et al., Science 271: 72-77, 1996. In an IgG1, the C-terminal Cys residue of the LC connects to the first HC Cys residue in the hinge; however, the LC and HC could still strongly associate together without the disulfide bond, as the association constant between them was estimated to be ˜10¹⁰ M⁻¹. Bigelow. C. et al., Biochemistry 13: 4602-4609, 1978; Horne, C. et al., J. Biol. Chem. 129: 660-664, 1982. Taken together, these observations suggest that the disulfide bonds in an IgG1 are vulnerable to certain attacks, and related cysteine residues could remain unpaired.

Reactive oxygen species (ROS) are a major cause of oxidative stress. ROS, such as hydrogen peroxides and alkyl hydroperoxides, can regulate the biological function of proteins. Poole, L. B. et al., Annu. Rev. Pharmacol. Toxicol. 44: 325-347, 2004; Philip, E., Free Rad. Biol. Med. 40: 1889-1899, 2006; Salmeen, A. et al., Nature 423: 769-773, 2003; Claiborne, A. et al., Adv. Protein Chem. 58: 215-276, 2001; Paget, M. S. B. and Buttner, M. J., Annu. Rev. Genet. 37: 91-121, 2003. Proteins that are regulated by H₂O₂ have characteristic cysteines, which are sensitive to oxidation because their environment promotes ionization of the thiol group (Cys-SH) to the thiolate anion (Cys-S⁻), which is more readily oxidized to sulfenic acid (Cys-SOH) than Cys-SH. Rhee, S. G. et al., (2000) Sci. STKE 2000, pel; Kim, J. R. et al., Anal. Biochem. 283: 214, 2000. The sulfenic acid is unstable and either reacts with any accessible thiol to form a disulfide or undergoes further oxidation to sulfinic acid (Cys-SO₂H) or sulfonic aid (Cys-SO₃H) Kice, J. L, Adv. Phys. Org. Chem. 17: 65, 1980; Claiborne, A., Biochemistry 38: 15407-15412, 1999.

Cysteine-based radicals can be formed by either short-range hydrogen atom abstraction or one-electron transfer reactions. Giles, N. M. et al., Chemistry & Biology 10: 677-693, 2003; Garrison, W. M., Chem. Rev., 87: 381-398, 1987; Bonifacic, M. et al., J. Chem. Soc. Pekin Trans., 2: 675-685, 1975; Elliot, A. J. et al., J. Phys. Chem. 85: 68-75, 1981; Jacob, C. et al., Biol. Chem. 387: 1385-1397, 2006. Thiyl (RS), sulfinyl (RSO), and sulfonyl (RSOO) radicals have been found to exist during oxidative stress. Harman, L. S. et al., J. Biol. Chem. 259: 5606-5611, 1984; Giles, G. I. and Jacob, C., Biol. Chem. 383: 375-388, 2002; Witting, P. K., and Mauk, A. G., J. Biol. Chem. 276: 16540-16547, 2001; Stadtman. E. R. and Levine, R. L., Amino Acids. 25: 207-218, 2003; Berlett, B. S. and Stadtman, E. R., J. Biol. Chem. 272: 20313-20316, 1997. Electron transfer between a Cys radical and other residues has been determined to be responsible for oligomeric product formation of myoglobin (Witting, P. K. and Mauk, A. G., J. Biol. Chem. 276: 16540-16547, 2001) while Pro and His residues were found to be the targets for ROS attacks that resulted in fragmentation of BSA and collagen. Garrison, W. M., Chem. Rev. 87: 381-398, 1987; Davies, M. J. and Dean, R. T., 1997, Radical mediated protein oxidation. Oxford University press, pp 50-120; Zhang, N. et al., J. Phys. Chem. 95: 4718-4722, 1991; Zhang, H. et al. J. Biol. Chem. 280: 40684-40698, 2005; Uchida, K. and Kawakishi, S., Biochem. Biophys. Res. Commun. 138: 659-665, 1986; Dean, R. T. et al., Free Radical Res. Commun. 7: 97-103, 1989. However, it remains unclear whether Cys-based radicals are involved in the cleavage of peptide bonds. Stamler and Hausladen (Stamler, J. S. and Hausladen A., Nat. Struct. Biol. 5: 247-251, 1998) have proposed a continuum of H₂O₂-mediated modifications that constitute important biological signaling events on the one hand and irreversible hallmarks of oxidative stress on the other.

Many different physiological and environmental processes lead to the formation of reactive oxygen species (ROS) in vitro and in vivo. The level of ROS in a cell depends on its age and physiological conditions and is a function of factors such as proteases, vitamins (A, C, and E) and redox metal ions. Bigelow, C. et al., Biochemistry 13: 4602-4609, 1978. Mitochondria are a significant source of ROS generation in cells. Salmeen, A. et al., Nature 423: 769-773, 2003. The rate of H₂O₂ production in isolated mitochondria is about 2% of the total oxygen uptake under physiological conditions. Salmeen, A. et al., Nature 423: 769-773, 2003; Claiborne, A. et al., Adv. Protein Chem. 58: 215-276, 2001; Paget, M. and Buttner, M., Annu. Rev. Genet. 37: 91-121, 2003.

ROS can lead to radical-mediated fragmentation and aggregation of proteins in vitro as well as in vivo. These oxidative modifications can reduce manufacturing yield of therapeutic and diagnostic products as well as reduce their efficacy. Antibodies have proven to be a particularly useful class of therapeutic and diagnostic proteins. However, the Fc hinge region of antibodies is prone to oxidative modification. This vulnerability to radical attack makes stabilization of the Fc hinge region a priority for the therapeutic and diagnostic development of antibody candidates as well as Fc-conjugated compounds in general.

### SUMMARY OF THE INVENTION

The present invention provides an immunoglobulin Fc comprising a hinge sequence of the IgG1 or IgG3 class which is resistant to radical-mediated fragmentation. Fragmentation resistance is manifested in a reduction in disulfide bond cleavage which would otherwise result in two half-antibodies, as well as a reduction in fragmentation events within the polypeptides making up each of these half antibodies. In one embodiment, the invention is an Fc-conjugate wherein the Fc is a human IgG1 or IgG3 Fc. The IgG1 and IgG3 Fc comprise a hinge core sequence which in one-letter amino acid code is THTCPXCP, wherein X represents an R or P residue. In the present invention, the H (histidine) residue in the hinge core sequence of native IgG1 or IgG3 Fc is substituted with a Ser (serine), Gln (glutamine), Asn (asparagine), or Thr (threonine) residue. In some embodiments the Fc-conjugate is in a pharmaceutically acceptable carrier.

The present invention is also directed to an isolated nucleic acid comprising a polynucleotide encoding the Fc or the Fc-conjugate of the present invention, as well as an expression vector comprising the isolated nucleic acid, and a host cell comprising the aforementioned expression vector. Thus, the present invention also includes compositions and methods of making the Fc or Fc-conjugate of the invention which can entail culturing in a suitable host cell the expression vector comprising the nucleic acid of the invention under conditions suitable to express the nucleic acid, and isolating the expressed Fc or Fc-conjugate from the host cell.

### BRIEF DESCRIPTION OF THE FIGURES

FIG. 1 shows the extent of radical mediated fragmentation of an IgG1 antibody resulting from H₂O₂ in combination with an additional reagent as detailed in the Examples.

FIG. 2 shows the extent of radical mediated fragmentation measured in milli-Absorbance Units (mAU) from inter-chain disulfide bond cleavage of various IgG1 hinge sequence substitution variants as detailed in the Examples.

### DETAILED DESCRIPTION OF THE INVENTION

The present invention provides compositions and methods relating to human IgG1 and IgG3 Fc and Fc-conjugates which are modified to be more resistant to radical-mediated fragmentation than native IgG1 or IgG3 Fc. These fragmentation resistant IgG1 and IgG3 Fc can be used in, e.g., the production of antibodies for therapeutic and diagnostic use having greater resistance to in vitro or in vivo fragmentation or aggregation. Compositions of the invention include: Fc-conjugates, polynucleotides comprising nucleic acids encoding the Fc or Fc-conjugates of the invention, vectors comprising these nucleic acids, host cells comprising and host cells expressing these vectors, and pharmaceutical compositions. Methods of making, and using, each of these compositions are also provided.

Units, prefixes, and symbols may be denoted in their SI accepted form. Unless otherwise indicated, nucleic acids are written left to right in 5′ to 3′ orientation; amino acid sequences are written left to right in amino to carboxy orientation. Numeric ranges recited herein are inclusive of the numbers defining the range and include and are supportive of each integer within the defined range. Amino acids may be referred to herein by either their commonly known three letter symbols or by the one-letter symbols recommended by the IUPAC-IUBMB Nomenclature Commission. Nucleotides, likewise, may be referred to by their commonly accepted single-letter codes. Unless otherwise noted, the terms “a” or “an” are to be construed as meaning “at least one of”. The section headings used herein are for organizational purposes only and are not to be construed as limiting the subject matter described.

### A. Definitions

As used herein, the term “antibody” includes reference to both glycosylated and non-glycosylated immunoglobulins of any isotype or subclass, including human (e.g., CDR-grafted), humanized, chimeric, multi-specific, monoclonal, polyclonal, and oligomers thereof, irrespective of whether such antibodies are produced, in whole or in part, via immunization, through recombinant technology, by way of in vitro synthetic means, or otherwise. Thus, the term “antibody” in inclusive of those that are prepared, expressed, created or isolated by recombinant means, such as (a) antibodies isolated from an animal (e.g., a mouse) that is transgenic for human immunoglobulin genes or a hybridoma prepared therefrom, (b) antibodies isolated from a host cell transfected to express the antibody (e.g., from a transfectoma), (c) antibodies isolated from a recombinant, combinatorial antibody library, and (d) antibodies prepared, expressed, created or isolated by any other means that involve splicing of immunoglobulin gene sequences to other DNA sequences. Such antibodies have variable and constant regions derived from germline immunoglobulin sequences of two distinct species of animals. In certain embodiments, however, such antibodies can be subjected to in vitro mutagenesis (or, when an animal transgenic for human immunoglobulin sequences is used, in vivo somatic mutagenesis) and thus the amino acid sequences of the V_{H} and V_{L} regions of the antibodies are sequences that, while derived from and related to the germline V_{H} and V_{L} sequences of a particular species (e.g., human), may not naturally exist within that species' antibody germline repertoire in vivo.

As used herein, “conjugate” means any chemical or biological moiety that, when conjugated to an Fc serves a diagnostic or therapeutic function. The conjugate can be directly or indirectly (i.e., through a chemical spacer) covalently attached. Exemplary conjugates include: cytotoxic or cytostatic agents (e.g., anti-tumor or anti-angiogenic agents), polyethylene glycol, lipids, and receptor or receptor fragments such as the extracellular domain of a cell-surface receptor.

A “host cell” is a cell that can be used to express a nucleic acid, e.g., a nucleic acid of the present invention. A host cell can be a prokaryote, for example, E. coli, or it can be a eukaryote, for example, a single-celled eukaryote (e.g., a yeast or other fungus), a plant cell (e.g., a tobacco or tomato plant cell), an animal cell (e.g., a human cell, a monkey cell, a hamster cell, a rat cell, a mouse cell, or an insect cell) or a hybridoma. Examples of host cells include the COS-7 line of monkey kidney cells (ATCC CRL 1651) (see Gluzman et al., Cell 23: 175, 1981), L cells, C127 cells, 3T3 cells (ATCC CCL 163), Chinese hamster ovary (CHO) cells or their derivatives such as Veggie CHO and related cell lines which grow in serum-free media (see Rasmussen et al., Cytotechnology 28: 31, 1998) or CHO strain DX-B11, which is deficient in DHFR (see Urlaub et al., Proc. Natl. Acad. Sci. USA 77: 4216-4220, 1980).

Typically, a host cell is a cultured cell that can be transfected with a polypeptide-encoding nucleic acid, which can then be expressed in the host cell. The phrase “recombinant host cell” can be used to denote a host cell that has been transfected with a nucleic acid to be expressed. Typically, a host cell comprises the nucleic acid but does not express it at an appreciable level unless a regulatory sequence is introduced into the host cell such that the regulatory sequence becomes operably linked with the nucleic acid. It is understood that the term host cell refers not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

The term “human antibody” refers to an antibody in which both the constant regions and the framework consist of fully or substantially human sequences such that the human antibody elicits substantially no immunogenic reaction against itself when administered to a human host and preferably, no detectable immunogenic reaction.

The term “humanized antibody” refers to an antibody in which substantially all of the constant region is derived from or corresponds to human immunoglobulins, while all or part of one or more variable regions is derived from another species, for example a mouse.

As used herein, “isolated” in the context of a nucleic acid means DNA or RNA which as a result of direct human intervention: 1) is integrated into a locus of a genome where it is not found in nature, 2) is operably linked to a nucleic acid to which it is not operably linked to in nature, or, 3) is substantially purified (e.g., at least 70%, 80%, or 90%) away from cellular components with which it is admixed in its native state.

The term “isolated” in the context of an Fc or Fc-conjugate means: (1) is substantially purified (e.g., at least 60%, 70%, 80%, or 90%) away from cellular components with which it is admixed in its expressed state such that it is the predominant species present, (2) is conjugated to a polypeptide or other moiety to which it is not linked in nature, (3) does not occur in nature as part of a larger polypeptide sequence, (4) is combined with other chemical or biological agents having different specificities in a well-defined composition, or (5) comprises a human engineered sequence not otherwise found in nature.

The terms “monoclonal antibody” or “monoclonal antibody composition” as used herein refer to a preparation of antibody molecules of single molecular composition, typically encoded by the same nucleic acid molecule. A monoclonal antibody composition displays a single binding specificity and affinity for a particular epitope. In certain embodiments, monoclonal antibodies are produced by a single hybridoma or other cell line (e.g., a transfectoma), or by a transgenic mammal. The term “monoclonal” is not limited to any particular method for making an antibody.

As used herein, “nucleic acid” and “polynucleotide” includes reference to a deoxyribonucleotide or ribonucleotide polymer, or chimeras thereof, and unless otherwise limited, encompasses the complementary strand of the referenced sequence.

A nucleic acid sequence is “operably linked” to a regulatory sequence if the regulatory sequence affects the expression (e.g., the level, timing, or location of expression) of the nucleic sequence. A “regulatory sequence” is a nucleic acid that affects the expression (e.g., the level, timing, or location of expression) of a second nucleic acid. Thus, a regulatory sequence and a second sequence are operably linked if a functional linkage between the regulatory sequence and the second sequence is such that the regulatory sequence initiates and mediates transcription of the DNA sequence corresponding to the second sequence. Examples of regulatory sequences include promoters, enhancers and other expression control elements (e.g., polyadenylation signals). Further examples of regulatory sequences are described in, for example, Goeddel, 1990, Gene Expression Technology: Methods in Enzymology 185, Academic Press, San Diego, Calif. and Baron et al., Nucleic Acids Res. 23: 3605-3606, 1995.

The terms “peptide,” “polypeptide” and “protein” are used interchangeably throughout and refer to a molecule comprising two or more amino acid residues joined to each other by peptide bonds. The terms “polypeptide”, “peptide” and “protein” are also inclusive of modifications including, but not limited to, glycosylation, lipid attachment, sulfation, gamma-carboxylation of glutamic acid residues, hydroxylation and ADP-ribosylation.

The terms “polynucleotide,” “oligonucleotide” and “nucleic acid” are used interchangeably throughout and include DNA molecules (e.g., cDNA or genomic DNA), RNA molecules (e.g., mRNA), and hybrids thereof. The nucleic acid molecule can be single-stranded or double-stranded.

As used herein, “specifically binds” or “specifically binding” or “binds specifically” refers to a binding reaction which is determinative of the presence of the target (e.g., a protein) in the presence of a heterogeneous population of proteins and other biologics. Thus, under designated immunoassay conditions, the specified Fc-conjugates such as antibodies or peptibodies, or other binding polypeptides bind to a particular protein and do not bind in a statistically significant amount to other proteins present in the sample. Typically, Fc-conjugates (e.g., antibodies, peptibodies) are selected for their ability to specifically bind to a protein by screening methods (e.g., phage display) or by immunization using the protein or an epitope thereof. See, Harlow and Lane (1998), Antibodies, A Laboratory Manual, Cold Spring Harbor Publications, New York, for a description of immunoassay formats that can be used to determine specific binding. For example, solid-phase ELISA immunoassays can be used to determine specific binding. Specific binding proceeds with an association constant of at least about 1×10⁷ M⁻¹, and often at least 1×10⁸ M⁻¹, 1×10⁹ M⁻¹, or, 1×10¹⁰ M⁻¹.

As used herein, “vector” includes reference to a nucleic acid used in the introduction of a polynucleotide of the present invention into a host cell. Vectors are often replicons. Expression vectors permit transcription of a nucleic acid inserted therein when present in a suitable host cell or under suitable in vitro conditions.

### B. Fc-Conjugates

The present invention provides isolated IgG1 and IgG3 Fc and Fc-conjugates, and methods of making and using these compositions, that are resistant to fragmentation and/or aggregation relative to a native IgG1 or IgG3 Fc. While not being bound by theory, the mechanism of free radical-mediated fragmentation has implicated a histidine residue present in the hinge core sequence of IgG1 immunoglobulins in fragmentation of the Fc. Appropriate substitution or deletion of that hinge core sequence histidine residue in an IgG1 and IgG3 Fc can reduce the degree of radical-mediated fragmentation and/or aggregation relative to an unmodified Fc or Fc-conjugate.

The present invention provides isolated Fc and Fc-conjugates having a modification rendering it resistant to fragmentation and/or aggregation from reactive oxygen species. The Fc (fragment crystallizable) of a mammalian immunoglobulin is a well characterized structure comprising a hinge region having a “hinge core sequence.” Table 1 shows a list of hinge core sequences, presented in one-letter amino acid code, found in human IgG subtypes. In the numbering system of Edelman et al. (Proc. Natl. Acad. Sci. USA 63: 78-85, 1969) the hinge core sequence of IgG1 corresponds to the IgG1 heavy chain residues 216-230 while the hinge core sequence of IgG3 corresponds to the IgG3 heavy chain residues 214-230. In the present invention, the histidine residue (“H”) present in the IgG1 or IgG3 hinge core sequence (at residue 224) as presented in Table 1 is substituted with a polar amino acid residue which is able to form hydrogen bonds. Specific examples of amino acid residues substitutable for the histidine residue in the hinge core sequence of IgG1 and IgG3 are Ser, Gln, Asn, or Thr residues. Alternatively, the histidine residue is deleted from the hinge core sequence.

TABLE 1
Sequence of the hinge core of IgG subtypes.

| IgG subtype | Hinge Core Sequence |
|---|---|
| IgG1 | EPKSCDKTHTCPPCP (SEQ ID NO: 1) |
| IgG2 | ERKCCVECPPCP (SEQ ID NO: 2) |
| IgG3 | ELKTPLGDTTHTCPRCP (SEQ ID NO: 3) |
| IgG4 | ESKYGPPCPSCP (SEQ ID NO: 4) |

The motif CPxCP is underlined.

Typically, the Fc of the Fc-conjugate of the present invention that is subject to the substitution or deletion yielding a radical-mediated fragmentation resistant Fc will be a human IgG1 or IgG3 Fc. However, a limited number of substitutions, additions, or deletions to a human IgG1 or IgG3 Fc can be made while retaining the properties of the IgG subtype. Thus, for example, 0, 1, 2, 3, 4, 5, 6, 7, 8, 9, or 10 amino acids of the IgG1 or IgG3 Fc can be modified and still be within the scope of the present invention. Thus, a modified IgG1 or IgG3 Fc will be 95%, 96%, 97%, 98%, or 99% identical to a native human IgG1 or IgG3 Fc. In some embodiments, the sole modification to the IgG1 or IgG3 hinge core sequence of the present invention (as presented in Table 1) is a substitution of the histidine residue in the hinge core sequence as described above. The Fc-conjugate can be monovalent or of a bivalent structure. Each conjugate of a bivalent Fc-conjugate can be the same or a different conjugate.

The conjugate that is covalently or non-covalently bonded to the Fc to form the Fc-conjugate can comprise or consist of a drug such as a chemotherapeutic compound, a diagnostic label such as a radiolabel, or a protein such as the extracellular domain of a human cell-surface receptor. In some embodiments the conjugate comprises or consists of an Fab antibody segment such that the Fc-conjugate is an IgG1 or IgG3 antibody. The antibody can be polyclonal or monoclonal. In some embodiments the Fc-conjugate is a fully human monoclonal, or a humanized monoclonal with CDR (complementarity determining regions) grafted from a non-human source (e.g., murine) onto an otherwise fully human IgG1 or IgG3. The antibody can be an agonistic or antagonistic antibody such that it activates or inhibits activation of a receptor. In some embodiments, that receptor is a human cell-surface receptor wherein the Fc-conjugate specifically binds to the extracellular domain of the cell-surface receptor. In other embodiments, the Fc-conjugate specifically binds to a ligand of a human cell-surface receptor such that it prevents binding of the ligand to the receptor. Examples of human cell-surface receptors to which the Fc-conjugates can bind include death receptor 4 (TRAIL Receptor-1), death receptor 5 (TRAIL Receptor-2), VEGF (vascular endothelial growth factor) receptor, a TNFR (tumor necrosis factor receptor), RANK (receptor activator nuclear factor kappa b) receptor, or Tie-1 and Tie-2 receptors. In other embodiments, the conjugate of the Fc-conjugate is a peptide (a “peptibody”) that specifically binds to a desired target. Peptibodies are taught in the International Application having publication number WO 2000/24782 (incorporated herein by reference).

### C. Nucleic Acids

The present invention is also directed to an isolated polynucleotide comprising a nucleic acid encoding the Fc of the Fc-conjugates of the present invention. Conveniently, when the conjugate of the Fc-conjugate is a protein (an “Fc-protein conjugate”) and encodes, e.g., an antibody, peptibody, or Fc-cell-surface receptor fusion (or fragment thereof), a nucleic acid of the present invention can encode the Fc-protein conjugate in its entirety.

Recombinant methods for producing the Fc and Fc-protein conjugates of the present invention commonly employ a polynucleotide comprising an isolated nucleic acid encoding the IgG1 or IgG3 Fc of the present invention. A nucleic acid encoding an Fc-protein conjugate of the invention can be directly synthesized by methods of in vitro oligonucleotide synthesis known in the art. Alternatively, smaller fragments can be synthesized and joined to form a larger fragment using recombinant methods known in the art. In some embodiments, nucleic acids primers with the desired hinge core sequence substitution or deletion are employed in PCR based in vitro mutagenesis to create the Fc or Fc-conjugates of the present invention. The polynucleotides of the present invention can also be constructed via in vitro synthetic means (e.g., solid phase phosphoramidite synthesis), or combinations thereof. Such methods are well known to those of ordinary skill in the art. See, for example, Current Protocols in Molecular Biology, Ausubel, et al., Eds., Greene Publishing and Wiley-Interscience, New York (1995).

### D. Construction of Fc-Conjugates

To express the isolated Fc or Fc-protein conjugates of the present invention, isolated DNA encoding these compositions can be obtained by standard molecular biology techniques (e.g., PCR amplification, site directed mutagenesis) and can be inserted into expression vectors such that the genes are operatively linked to transcriptional and translational regulatory sequences.

The present invention thus includes expression vectors (polynucleotides) comprising nucleic acids of the present invention. Expression vectors include plasmids, retroviruses, cosmids, YACs, EBV derived episomes, and the like. The expression vector can encode a signal peptide that facilitates secretion of the Fc or Fc-protein conjugate of the present invention from a host cell. The Fc or Fc-protein conjugate gene can be cloned into the vector such that the signal peptide is linked in-frame to the amino terminus of the Fc/Fc-protein conjugate gene. The signal peptide can be an immunoglobulin signal peptide or a heterologous signal peptide (i.e., a signal peptide from a non-immunoglobulin protein).

The expression vector and expression control sequences are chosen to be compatible with the expression host cell used. A compatible vector and host cell system can allow, for example, co-expression and assembly of the variable heavy and variable light chains of an Fc-conjugate which is an antibody. Suitable systems for expression can be determined by those skilled in the art. In some embodiments, the expression vectors are split DHFR vectors, PDC323 or PDC324; see, McGrew, J. T. and Bianchi, A. A. (2002) “Selection of cells expressing heteromeric proteins”, U.S. Patent Application No. 20030082735; and, Bianchi, A. A. and McGrew, J. T., “High-level expression of full antibodies using trans-complementing expression vectors,” Bioengineering and Biotechnology 84(4): 439-444, 2003. When the Fc-conjugate is an antibody, the variable heavy chain nucleic acid and the antibody variable light chain nucleic acids of the present invention can be inserted into separate vectors or, frequently, both genes are inserted into the same expression vector. The nucleic acids can be inserted into the expression vector by standard methods (e.g., ligation of complementary restriction sites on the antibody nucleic acid fragment and vector, or blunt end ligation if no restriction sites are present).

Nucleic acids and expression vectors of the present invention can be introduced into a host cell via transfection. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the Fc-conjugates of the invention in either prokaryotic or eukaryotic host cells, expression of antibodies in eukaryotic cells, and most preferably mammalian host cells, is the most typical because such eukaryotic cells, and in particular mammalian cells, are more likely than prokaryotic cells to assemble and secrete a properly folded and immunologically active antibody.

The expression vectors of the invention carry regulatory sequences that control the expression of the sequence in a host cell. Such regulatory sequences are described, for example, in Goeddel; Gene Expression Technology. Methods in Enzymology 185, Academic Press, San Diego, Calif. (1990). It will be appreciated by those skilled in the art that the design of the expression vector, including the selection of regulatory sequences may depend on such factors as the choice of the host cell to be transformed, the level of expression of protein desired, and the like. Preferred regulatory sequences for mammalian host cell expression include viral elements that direct high levels of protein expression in mammalian cells, such as promoters and/or enhancers derived from cytomegalovirus (CMV), Simian Virus 40 (SV40), adenovirus, (e.g., the adenovirus major late promoter (AdMLP)) and polyoma. Alternatively, nonviral regulatory sequences may be used, such as the ubiquitin promoter or beta-globin promoter.

The expression vectors of the invention may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr-host cells with methotrexate selection/amplification) and the neo gene (for G418 selection).

Preferred mammalian host cells for expressing the Fc or Fc-conjugates of the invention include Chinese Hamster Ovary (CHO cells) (including dhfr-CHO cells, described in Urlaub and Chasin, Proc. Natl. Acad. Sci. USA 77: 4216-4220, 1980, used with a DHFR selectable marker, e.g., as described in Kaufman, R. J. and Sharp, P. A., Mol. Biol. 159: 601-621, 1982), NS/0 myeloma cells, COS cells and SP2.0 cells. In particular for use with NS/0 myeloma cells, another preferred expression system is the GS gene expression system disclosed in WO 87/04462, WO 89/01036 and EP 338841. When expression vectors of the invention are introduced into mammalian host cells, the Fc or Fc-conjugates are produced by culturing the host cells in the appropriate culture media for a period of time sufficient to allow for their expression in the host cells or, more preferably, secretion of the Fc or Fc-conjugate into the culture medium in which the host cells are grown.

Once expressed, the Fc or Fc-conjugate can be purified for isolation according to standard methods in the art, including HPLC purification, fraction column chromatography, gel electrophoresis and the like (see, e.g., Scopes, Protein Purification, Springer-Verlag, NY, 1982). In certain embodiments, polypeptides are purified using chromatographic and/or electrophoretic techniques. Exemplary purification methods include, but are not limited to, precipitation with ammonium sulphate; precipitation with PEG; immunoprecipitation; heat denaturation followed by centrifugation; chromatography, including, but not limited to, affinity chromatography (e.g., Protein-A-Sepharose), ion exchange chromatography, exclusion chromatography, and reversed-phase chromatography; gel filtration; hydroxylapatite chromatography; isoelectric focusing; polyacrylamide gel electrophoresis; and combinations of such and other techniques. In certain embodiments, a polypeptide is purified by fast protein liquid chromatography or by high performance liquid chromotography (HPLC).

### E. Pharmaceutical Compositions

The present invention provides pharmaceutical compositions comprising Fc and Fc-conjugates of the present invention formulated with a pharmaceutically acceptable carrier. In some embodiments, the pharmaceutically acceptable carrier is suitable for administration in human subjects. As used herein, “pharmaceutically acceptable carrier” includes any and all solvents, dispersion media, coatings, antibacterial and antifungal agents, isotonic and absorption delaying agents, and the like that are physiologically compatible when administered to a particular subject. Pharmaceutical compositions typically must be sterile and stable under the conditions of manufacture and storage.

Pharmaceutical compositions of the invention can be administered in combination therapy, i.e., combined with other agents. Agents are inclusive of, but not limited to, in vitro synthetically prepared chemical compositions, antibodies, antigen binding regions, radionuclides, and combinations and conjugates thereof.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

### F. Therapeutic and Diagnostic Conjugates

The various therapeutic moieties described herein that improve the therapeutic and/or diagnostic benefit can be covalently linked, directly or indirectly (e.g., via a “linking group”) to an Fc of the present invention to yield an Fc-conjugate. A linking group is optional. The linker is often made up of amino acids linked together by peptide bonds. One or more of these amino acids may be glycosylated, as is well understood by those in the art. Non-peptide linkers are also possible. An exemplary non-peptide linker is a PEG (polyethylene glycol) linker.

Techniques for conjugating such therapeutic moieties to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-256 (Alan R. Liss, Inc. 1985); Hellstrom et al., “Antibodies For Drug Delivery”, in Controlled Drug Delivery (2nd Ed.), Robinson et al. (eds.), pp. 623-653 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-316 (Academic Press 1985), and Thorpe et al., “The Preparation And Cytotoxic Properties Of Antibody-Toxin Conjugates”, Immunol. Rev. 62: 119-158, 1982. The compositions of the present invention can be coupled to radionuclides, such as 1311, 90Y, 105Rh, indium-111, etc., as described in Goldenberg, D. M. et al. Cancer Res. 41: 4354-4360, 1981, and in EP 0365 997.

### EXAMPLES

The following examples, including the experiments conducted and results achieved, are provided for illustrative purposes only and are not to be construed as limiting the present invention.

### Example 1

This example describes the results of a specific hinge fragmentation of a human IgG1 antibody by H₂O₂-mediated radical cleavage that led to the loss of one Fab domain and the formation of a partial molecule. H₂O₂ attack of the IgG1 resulted in the breakage of the inter-chain disulfide bond between the two cysteine residues located at position 226 (Cys²²⁶) in the hinge region and followed by the formation of sulfenic acid (Cys²²⁶SOH) and a thiyl radical (Cys²²⁶S*), which initializes an electron transfer to upper hinge residues, leading to radical-mediated polypeptide backbone fragmentation.

The antibody used was a recombinant fully human antibody of the IgG1 subclass. The molecule was expressed in CHO cells and chromatographically purified using conventional techniques. The antibody fragments were separated by size exclusion chromatography (SEC). The cleavage of antibody was measured by a percentage of partial molecules (C1 and C2).

Briefly, a reaction mixture (1.0 mL) containing 2 mg to 10 mg of IgG1 antibody in a buffer was incubated with varying concentrations of H₂O₂. To remove H₂O₂, the samples were buffer exchanged by centrifugation in filter units. Purified partial molecules (˜1 mg/mL) were reduced and alkylated. The alkylation was performed at room temperature in the dark and a 0.5 M DTT stock solution was added to quench the alkylation. Reversed-phase high-performance liquid chromatography (RP-HPLC) was performed followed by electrospray ionization (ESI) time-of-flight (TOF) mass spectrometry (MS).

Purified bulk antibody was analyzed by size exclusion chromatography (SEC), and showed ˜0.9% of a partial molecule (P1). This is not a single case from one lot but was present in several runs with a range of 0.9-1.1%. The P1 specie was further purified by SEC to purity greater than 95%, and analyzed by RP-HPLC-TOF/MS. The results indicated that P1 is a heavily oxidized partial antibody that lost one Fab domain.

H₂O₂ is known to be capable of causing oxidation and damage to proteins. To explore if oxidative stress caused the cleavage, H₂O₂ was employed to treat the IgG1, and the impact was measured by SEC. Over the range of 5-20 mM H₂O₂, no notable cleavage was found for the first 8 hours of incubation. Only after 48 hours of incubation with 20 mM H₂O₂, two partial fragments—C1 and C2—were observed. The amounts of these two fragments grew in direct proportion to the length of incubation. This fragmentation is also dependent on the antibody concentration and pH conditions. In addition, the cleavage proceeded without a significant steady phase even up to 8 weeks. The fact that only two products (C1 and C2) were observed suggested that the cleavage was specific and probably driven by a specific mechanism. Subsequent work demonstrating the heavily oxidized nature of P1 suggests that the hinge fragmentation may result from oxidative stress during CHO cell production of the antibody. The similarity between P1 and C1, particularly the higher oxidation levels observed with prolonged H₂O₂ treatment, suggests that oxidative stress caused the hinge fragmentation.

RP-HPLC-TOF/MS analysis of the C1 and P1 showed that they were the same species, each of them was a heavily oxidized partial molecule missing a Fab domain, in particular, the single complementary HC of the Fc domain comprised a unique “ladder” of the N-terminal residues Asp²²¹, Lys²²², Thr²²³, and Thr²²⁵ in the upper hinge region. In addition, two adducts of 45 Da and 71 Da were observed in some Fc fragments, these are not common adducts as they are not consistent with known modifications.

RP-HPLC-TOF/MS analysis of the C2 fragment revealed that it is the Fab domain of the IgG1, and is heavily oxidized. The LC of C2 displayed a similar profile to its counterpart in C1. The Fab portion of the HC (Fd) in C2 had two components, both of which were heavily oxidized with one or three oxygen additions. The more highly oxidized component contained a ladder of C-terminal residues Asp²²¹, Lys²²², Thr²²³, His²²⁴, and Thr²²⁵; the more lightly oxidized Fd component possessed a wider ladder, consisting of C-terminal residues from Ser²¹⁸ to Thr²²⁵. These results indicated that H₂O₂ treatment resulted in hinge cleavage and significant level of oxidation in both the LC and HC of the IgG1.

Combining the nature of these adducts and their locations, the data suggests that radical cleavage was responsible for the hinge fragmentation. Hydrogen peroxides can regulate the biological function of proteins through radical induced oxidation pathways. Reaction with hydroxyl radicals could result in various chemical reactions that lead to the degradation of a protein (Garrison, W. M., Chem. Rev. 87: 381-398, 1987; Davies, M. J. and Dean, R. T., 1997, Radical mediated protein oxidation. Oxford University press, pp 50-120; Berlett, B. S. and Stadtman, E. R., J. Biol. Chem. 272: 20313-20316, 1997).

To examine if OH radicals are involved in the hinge fragmentation and to evaluate some factors that may influence the cleavage, the IgG1 was subjected to H₂O₂ attack after some pretreatments. These include N-ethyl-maleimide (NEM) pretreatment to block unpaired Cys residues prior to H₂O₂ treatment, or adding catalase or ethylene-diamine-tetra-acetic acid (EDTA) into the reaction system. SEC was performed to measure the impact. It was found that catalase almost completely blocked cleavage, strongly indicating that the OH radicals were important for cleavage. Total free thiol groups were measured to be ˜0.28 mol/mol antibody under denatured conditions in the presence of 4 M GdnHCl using Ellman's reagent, 5,5′-dithiobis (2-nitrobenzoic acid) (DTNB). Prior to H₂O₂ treatment, the IgG1 was incubated with NEM at pH 5.0 for 3 hours at 37° C. The NEM blocked sample showed only a ˜7% decrease in cleavage, while the free thiol (—SH) groups were completely blocked by the NEM-treatment. Therefore, the results suggested that the unpaired Cys residues were not critical for the cleavage.

The spin trap 5,5-dimethyl-1-pyrroline N-oxide (DMPO) is widely used to provide evidence for the involvement of free radicals in many biological reactions, particularly for OH radicals. DMPO has been used to identify the radical sites exposed to radical damage in myoglobin and other molecules. Therefore, the IgG1 was treated with H₂O₂ in the presence of DMPO for one week, and the fragmentation was monitored by SEC. In a molar ratio range of 50:1 to 5:1 of DMPO:H₂O₂, DMPO completely blocked fragmentation over a time course of two weeks of incubation.

To identify the radical formation site, Lys-C peptide mapping was performed. While the Cys²³¹-SO₃H-containing intact hinge peptide was observed, only HC Cys²³¹ was found to contain the DMPO adduct. Finding no radical formation at upper hinge residues is unlikely due to mass spectrometry sensitivity issues or the reaction rates between OH radicals and upper hinge residues. It has been determined that OH radicals have a rate constant with Cys of 3.4×10¹⁰ M⁻¹s⁻¹, much faster than His (1.3×10¹⁰ M⁻¹s⁻¹), Thr (5.1×10⁸ M⁻s⁻¹), Asp (7.5×10⁷ M⁻¹s⁻¹), and Lys (3.5×10⁷ M⁻¹s⁻¹) (Davies, M. J. and Dean, R. T., 1997, Radical mediated protein oxidation. Oxford University press, pp 50-120). Therefore, these results demonstrated the necessity of an electron transfer from the HC Cys²³¹ to a residue in the upper hinge that led to a radical cleavage per molecule. It was also determined that an electron has a reaction rate constant with His of 6.4×10⁷ M ⁻¹s ⁻¹, Thr of 2.0×10⁷ M⁻¹s⁻¹, Lys of 2.0×10⁷ M⁻⁷s⁻¹, Asp of 1.8×10⁷ M⁻¹s⁻¹ (Davies, M. J. and Dean, R. T., 1997, Radical mediated protein oxidation. Oxford University press, pp 50-120), indicating that these residues are capable of localizing an electron to proceed to radical-induced backbone cleavage. This mechanism explains the specific hinge fragmentation that generated the complementary C-terminal residues in the Fab fragment (C2) and the N-terminal residues in the Fc of the partial antibody (C1).

### Example 2

This example summarizes the results of radical-mediated fragmentation of the IgG1 Fc.

1. IgG1 bulk antibody contains ˜1% of a truncated antibody (P1), which was determined to be a heavily oxidized form, with one of the Fab domains missing.
2. Reaction of H₂O₂ with IgG1 bulk drug substance (BDS) generated a truncated molecule and one free Fab domain fragment by specific cleavages in the hinge region which resulted in the formation of a C-terminal ladder of residues (Cys²²⁰-Asp²²¹-Lys²²²-Thr²²³-His²²⁴-Thr²²⁵) in the Fab domain of the heavy chain (Fd) and a complementary N-terminal ladder of residues in the Fc domain.
3. In the H₂O₂ treated samples, for the majority of intact and truncated molecules the inter-chain disulfide bond between the Cys²²⁶ residues was found to be intact.
4. In the BDS sample, there was no unpaired disulfide bond in the hinge region observed by the native Lys-C peptide map that was performed after pre-blocking any potential unpaired Cys by N-etheylmaleimide (NEM).
5. LC-MS/MS analysis identified a small amount of Cys-SO₃H at Cys²²⁶ in both the intact hinge peptide (THT Cys²²⁶PPCAPELLGGPSVFLFPPKPK) (SEQ ID NO:5) and the truncated hinge peptide (Cys²²⁶PPCAPELLGGPSVFLFPPKPK) (SEQ ID NO:6).
6. In the truncated antibody, adducts were identified in the N-terminal hinge region of the Fc domain as either isocyanate or N-α-ketoacyl derivatives that introduced an additional mass of 45 or 71 Da, respectively.
7. The IgG1 contains ˜0.28 mol/mol antibody unpaired Cys residues, which are not critical for the cleavage reaction as demonstrated by the fact that blocking all unpaired Cys residues caused no or only little effect on the fragmentation.
8. A widely used radical spin trap 5,5′-dimethyl-1-pyrroline N-oxide (DMPO) was found capable of blocking the hinge fragmentation because of its binding to Cys²²⁶. However, DMPO binding did not block the formation of Cys²²⁶-SO3H.

### Example 3

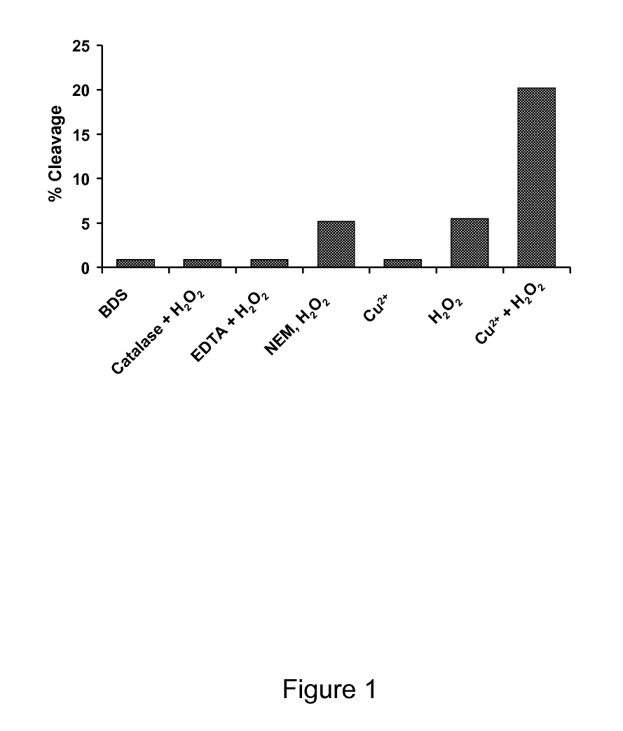

This example demonstrates that hydroxyl radicals and not Cu²⁺ induces hinge fragmentation. Hydrogen peroxides can regulate the biological function of proteins through radical induced oxidation pathways. Additionally, reaction with hydroxyl radicals can lead to various chemical reactions that result in the degradation of a protein. To examine if OH radicals are involved in the hinge fragmentation and to evaluate several factors that may influence the cleavage, the IgG1 was subjected to H₂O₂ attack. As shown in FIG. 1, the H₂O₂ induced fragmentation was completely blocked by catalase, indicating that OH radicals were responsible for the cleavage. Total free thiol groups were determined to be ˜0.28 mol/mol antibody under denatured conditions in the presence of 4 M GdnHCl using Ellman's reagent, 5,5′-dithiobis (2-nitrobenzoic acid) (DTNB). Prior to H₂O₂ treatment, the IgG1 was incubated with NEM at pH 5.0 for 3 hours at 37° C. The NEM blocked sample showed only a ˜7% decrease in cleavage, whereas the free thiol (—SH) groups were found completely blocked by the NEM-treatment suggesting that the unpaired Cys residues were not critical for the cleavage.

In addition, it was found that a pre-incubation with EDTA inhibited ˜90% of the H₂O₂ induced cleavage of the IgG1, suggesting an involvement of transition metals in the reaction. However, such pretreatment did not completely block the cleavage with H₂O₂ still capable of cleaving the IgG1, despite having a slower reaction rate. These results suggested that OH radicals are responsible for the hinge fragmentation, and that the reaction can be accelerated by a metal-catalyzed reaction to generate OH radicals. This hypthesis was supported by the observation that treatment with H₂O₂ in the presence of 10 μM of copper acetate (Cu(OAc)₂) resulted in approximately 4-times more cleavage than H₂O₂ treatment alone, whereas 10 μM Cu(OAc)₂ alone produced only little cleavage during a 5-day incubation.

Smith et. al. reported a cleavage of the K-T bond in the upper hinge DKTHT (SEQ ID NO:7) residues of an IgG1 (Smith, M. A. et al., Int. J. Pept. Protein Res., 48: 48-55, 1996) with 1 mM of CuSO₄ at neutral or basic pH by examining a number of synthetic peptides. Under the experimental conditions described here (pH 5.2 and incubation at 25° C.), the Cu²⁺ binding to the upper hinge residues (e.g., His, Lys) is less favorable than at neutral or basic pH, and resulted in a ˜30% increase of the hinge fragmentation. Since there are trace amounts of transition metal ions present in solvents or proteins, their concentration could be sufficient to function as a catalyst for the radical induced hinge fragmentation. This conclusion is also consistent with the theory that some transition metals (e.g., Cu²⁺ and Fe³⁺) play an important role in the site-selective radical attack either by binding to a protein or staying in solution. In both cases, the metal accelerates the reaction by catalyzing the generation of hydroxyl radicals through a Fenton-like reaction. Collectively, these facts independently confirmed a radical induced hinge fragmentation mechanism.

### Example 4

This example proposes a mechanism of radical-mediated Fc fragmentation. Our experimental results of studying a human IgG1 revealed a radical mediated hinge fragmentation in this human IgG1 antibody.

The trace amount of transition metal catalyzes the generation of OH radicals in the reaction system. Reaction of the IgG1 antibody with OH radicals resulted in the breakage of the inter-chain disulfide bond between the two cysteine residues located at position 226 (Cys²²⁶) in the hinge region (Cys²²⁶-Pro-Pro-Cys-Pro) of the antibody. The disulfide bond breakage was followed by the formation of sulfenic acid (Cys²²⁶-SOH) and a thiyl radical (Cys²²⁶-S*). Subsequent reactions of these species in the presence of oxygen resulted in the formation of sulfinic acid (Cys²²⁶-SO₂H) and sulfonic acid (Cys²²⁶-SO₃H) as the principal products. Meanwhile the thiyl radical initializes an electron transfer upstream, along the hinge polypeptide backbone. This electron transfer leads to radical-mediated polypeptide backbone fragmentation, which is characterized by a ladder of C-terminal residues in the Fab domain of the heavy chain (Fd), created due to cleavage at several neighboring hinge residues (Asp²²¹, Lys²²², Thr²²³, His²²⁴ and Thr²²⁵). We observed binding of 5,5′-dimethyl-1-pyrroline N-oxide (DMPO), a widely used radical spin trap, only at Cys²²⁶, which blocked the hinge fragmentation. The specific binding of DMPO to only Cys²²⁶ confirmed that the radical only exists at Cys²²⁶ in the CPPCP sequence, which is a highly conserved hinge sequence motif Cys-Pro-X-Cys-Pro (X=Pro, Arg and Ser) among IgG molecules (Table 1).

The determination of the +45 Da adduct suggested a radical cleavage mechanism that generated an isocyanate structure (MW=28 Da) at the N-terminus of Fc through the diamide pathway. Due to its unstable nature, the isocyanate group hydrolyses into carboxylic acid (the +45 Da adduct). On the other hand, OH radical attack at the γ-carbon position of the side chain of certain amino acids could result in oxidative degradation that leads to the formation of an unsaturated product of dehydropeptides, which only retains a β-CH₂ group as a side chain. This compound can be easily hydrolyzed to yield amide and keto acid functions, the +71 Da adduct (an N-pyruvyl group). To this end, the observed +71 Da adduct at the N-terminus of Thr²²⁵ could have been yielded from the oxidative degradation of His²²⁴. Meanwhile, hydrolysis of these unstable intermediates would be another way to recycle them, and this process resulted in some truncated hinge peptides that contain regular N-terminal residues. Taken together, the +45 Da and +71 Da adducts at the N-terminal residues of the upper hinge region are the products of radical cleavage at the α-carbon of the protein backbone and γ-carbon position of a amino acid side chain, respectively, confirming a radical mediated mechanism for protein backbone cleavage.

### Example 5

This example demonstrates the resistance to radical-mediated fragmentation by mutation of the His and Lys residues in the hinge core sequence. An investigation was conducted to determine the effect of mutating His²²⁴ and Lys²²² in comparison with the human wild-type IgG1. Wild type IgG1 and seven mutants were incubated with H₂O₂ and the formation of the partial molecule and in particular the release of the Fab domain fragment was monitored by SEC. The seven mutants were: Lys²²²Ser (K/S), Lys²²²Gln (K/Q), Lys²²²Ala (K/A), His²²⁴Ser (H/S), His²²⁴Gln (H/Q), His²²⁴Ala (H/A) and Lys²²²Ser/His²²⁴Ser (K/S+H/S). Among these mutants, replacing His with Gln or Ser almost totally blocked (>97%) OH radical induced fragmentation that led to a release of the Fab domain (C2) and the partial molecule C1. The His/Ala mutation showed ˜6% of fragmentation vs ˜15% for the native IgG1 over a 8-day incubation period. In contrast, all single Lys mutants promoted the cleavage by 31-33%. More importantly, the double mutant K/S+H/S showed a >97% inhibition of fragmentation, the same percentage measured for the single His/Ser or His/Gln mutant, indicating the importance of the His residue in the fragmentation.

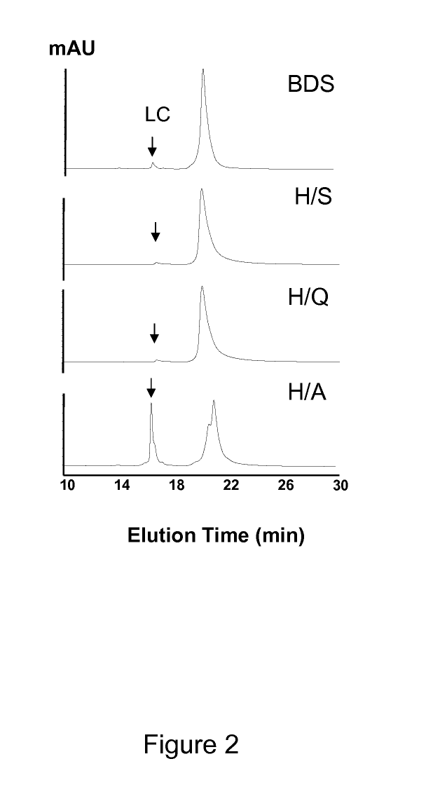

Although the His/Ala mutant showed cleavage, it was not known whether the mutant did comprise the same structural degradations. It had been documented that the LC and HC remain strongly associated without the inter-disulfide bond connecting them (Bigelow. C. et al., Biochemistry, 13: 4602-4609, 1978). Therefore, it is possible that the LC and HC are held together without the inter-disulfide bond and show a similar SEC profile as the Fab domain fragment. Therefore, the mutants were further examined by RP-HPLC-TOF/MS under non-reducing conditions after 1-day of H₂O₂ treatment. Under these conditions, it is expected that only non-covalently bonded components would be separated from the main species. As shown in FIG. 2, besides the main peak eluting at ˜21 minutes, one component, migrating with a retention time of 16.5 minutes, was observed for all mutants. In particular the H/A mutant released this specie approximately 15-times more than the H/S and H/Q mutants. TOF/MS analysis determined a molecular mass of 23,437.5 Da for this specie, which is +48 Da heavier than the theoretical mass of 23,389.0 Da for the LC. RP-HPLC-MS/MS analysis of the Lys-C peptide map confirmed that the specie showed full conversion of the LC Cys²¹⁵ to sulfonic acid (+48 Da), suggesting that the breakage of the inter-disulfide bond by H₂O₂ attack led to the oxidation of the LC. These results suggested that the removal of an OH group abolishes the capability of H-bond formation in the side chain and adversely impaired the ability of this residue to resist a radical attack.

By using a synthetic peptide (FDKTHTY) (SEQ ID NO:8), Allen et al. (Allen, G. and Campbell, R., Int. J. Peptide Protein Res. 48: 265-273, 1996) found that a His/Ala substitution prevented Cu²⁺(1 mM) induced cleavage of the peptide, which comprises the same sequence (DKTHT) (SEQ ID NO:7) as the upper hinge of an IgG1. However, our results clearly indicated that the His/Ala mutant did not prevent the release of the LC due to the H₂O₂ induced breakage of the inter-disulfide bond between the LC and HC. The loss of the LC would destroy the function of the IgG. Particularly the hinge region where the two hinge inter-disulfide bonds connect the two HC with the upper hinge (DKTHT) (SEQ ID NO:7) connecting to the Fab domain, is a double stranded structure that restrains the hinge to adopt a conformation that is most likely very different than the conformation of the synthetic peptide in solution. Consequently, results obtained from a peptide need to be taken with caution when applied to a protein that contains the same or similar sequence. Taken together, our results clearly indicated that the His/Ser and His/Gln mutants, but not the His/Ala mutant inhibited the OH radical mediated cleavage.

Given the nature of the side chains of His, Gln, Ser, Ala and Lys, the results of analyzing these mutants allowed us to conclude that the imidazole ring rather than the γ-carbon in the side chain of the His residue is responsible for the hinge cleavage. This hypothesis was supported by the observation that the His/Gln mutant inhibited the radical induced cleavage while Gln has a γ-carbon in its side chain. The major site of electron attachment appears to be at the imidazole ring of His²²⁴ at pH values where this is protonated. OH radicals are known to attach to the C-2, C-4 and C-5 position of the imidazole ring. Based on the positioning of these hinge residues in the known three-dimensional structure of the IgG1 and the hydrogen bond network around the hinge, we propose that subsequent addition of oxygen to these species made the initial radicals undergo base-catalyzed loss of water to give a highly stabilized bisallylic radical. The His residue functions as the central target to localize an electron, and subsequently extract protons from neighboring residues, led to radical induced cleavage by the diamide and α-amidation pathways. Taken together, the results demonstrated the feasibility of preventing hinge fragmentation using rational design.

What is claimed:

1. An isolated radical-mediated fragmentation resistant Fc-conjugate, wherein said Fc is a human IgG1 or IgG3 Fc and wherein the Fc comprises a hinge core sequence THTCPXCP (SEQ ID NO:9), wherein X is R or P, and wherein the H residue in said hinge core sequence is substituted with a Ser, Gln, Asn, or Thr residue.

2. The Fc-conjugate of claim 1, wherein said Fc-conjugate is a monoclonal antibody, peptibody, or Fc-receptor fusion.

3. The Fc-conjugate of claim 2, wherein the monoclonal antibody is a fully human monoclonal antibody.

4. The Fc-conjugate of claim 3, wherein histidine residue in said motif is a serine or glutamine residue.

5. The Fc-conjugate of claim 4 in a pharmaceutically acceptable carrier.

6. An isolated nucleic acid comprising a polynucleotide encoding the Fc-conjugate of claim 2.

7. An isolated expression vector comprising the isolated nucleic acid of claim 6.

8. A host cell comprising the expression vector of claim 7.

9. The host cell of claim 8, wherein the host cell is a CHO cell.

10. A method of making an Fc-conjugate of claim 2, comprising culturing in a suitable host cell the expression vector of claim 5 under conditions suitable to express the vector, and isolating the expressed Fc-conjugate from said host cell.


SEQUENCE LISTING (9 sequences)

SEQ ID NO: 1 - protein, 15 aa, Homo sapiens
EPKSCDKTHTCPPCP

SEQ ID NO: 2 - protein, 12 aa, Homo sapiens
ERKCCVECPPCP

SEQ ID NO: 3 - protein, 17 aa, Homo sapiens
ELKTPLGDTTHTCPRCP

SEQ ID NO: 4 - protein, 12 aa, Homo sapiens
ESKYGPPCPSCP

SEQ ID NO: 5 - protein, 25 aa, Homo sapiens
THTCPPCAPELLGGPSVFLFPPKPK

SEQ ID NO: 6 - protein, 22 aa, Homo sapiens
CPPCAPELLGGPSVFLFPPKPK

SEQ ID NO: 7 - protein, 5 aa, Homo sapiens
DKTHT

SEQ ID NO: 8 - protein, 7 aa, Homo sapiens
FDKTHTY

SEQ ID NO: 9 - protein, 8 aa, Homo sapiens
  misc_feature (6)..(6): X  can be Arginine or Proline
THTCPXCP